(19) United States
(12) Patent Application Publication
(10) Pub. No.: US 2012/0009587 A1
(43) Pub. Date: Jan. 12, 2012

(54) Recombinant adenovirus target-oriented co-expressing human p53 and p53aip1

(76) Inventors: Shangwu Wang, Guangdong (CN); Yanan Zhu, Guangdong (CN)

(21) Appl. No.: 12/310,710

(22) PCT Filed: Aug. 31, 2007
(86) PCT No.: PCT/CN2007/002609

§ 371 Date: Mar. 3, 2009

(30) Foreign Application Priority Data
Sep. 4, 2006 (CN) 200610037451.8

(51) Int. Cl. C12Q 1/68; C12N 7/01

(52) U.S. Cl. 435/6.15; 435/235.1

(57) ABSTRACT

A recombinant adenovirus target-oriented co-expressing human p53 variant and p53AIP1 is disclosed, which is inserted an expression cassette co-expressing human p53 variant and p53AIP1 in E1 deletion region of the adenovirus. The expression cassette consists of tumor specific promoter, human p53 variant, internal ribosome entry site (IRES), human p53AIP1, and SV40.

### BACKGROUND OF THE PRESENT INVENTION

1. Field of Invention

The present invention relates to an adenovirus, and more particularly to an adenovirus of target-oriented coexpressing p53 and p53AIP1 and producing method thereof.

2. Description of Related Arts

P53 gene is one of the main members of tumor suppressor gene, and the p53 gene is the most relevant gene of human tumor occurrence. Wild-type p53 gene plays an important role in prevention of occurrence and inhibition of proliferation of malignant tumor cells. The changes of environmental factors such as ultraviolet light, radiation, chemicals, and the metabolites produced by human body itself can lead to the cells' DNA damages. Under normal physiological circumstances, these damaged cells' DNA can be repaired or clean up by degradation to prevent the heredity of the damaged cells' DNA through effect of some genes, so as to make sure the replication of normal cells' DNA to protect the normal cell's functional operation. Otherwise, the damaged cells' DNA will keep replicating and separating along with the separation of chromosomes to result in a large number of DNA mutations and chromosomal aberrations of the chromosome sets. The aggregation of those mutations will make the normal cell malignant and develop into tumor cells finally.

At present a large number of researches show that wild-type p53 protein plays a biological function as “Gene Warrior” within cells to monitor the integrity and stability of cell genome. When cells' DNA is damaged, the p53 can rapidly activate transcription of p21 gene, and inhibit transcription of variety of proto-oncogene such as c-Fos, c-jun, Rb, and other related gene such as IL-6, PCNA to stagnate cell division at the G1 phase. Meanwhile, the wild-type p53 protein interacts with a replication factor (RPA) to involve in DNA replication and repair. More importantly, wild-type p53 protein can start process of cell apoptosis program and induce cell suicide to prevent malignant-tended cells further splitting and proliferating, so as to prevent occurrence of cancer.

In human malignant cells, p53 gene mutation is the most common genetic variation. According to a statistic, there are about 50% human tumor related to the p53 gene mutation, and p53 gene mutation exists in 30 to 40% of breast cancer, 50% of lung cancer, and 70% of colon cancer and in almost 100% of small cell lung cancer. Besides, animal experiments proved that tumor had occurred in 100 percentages of mice expressing abnormal p53 gene protein.

As so importance of its biological functions and its high relevant to occurrence of human tumor and to malignancy of cancers, scientists have applied the biological functions of exogenous wild-type of p53 gene to induce apoptosis process of tumor cells with p53 gene mutation in the experimental researches and clinical gene therapies studies of tumor since 1980. Now a lot of research reports and clinical studies results have proved that gene therapy of wild-type p53 was safe and effective, and only had negligible side effects.

The results of these clinical studies show that wild-type p53 gene has relatively better therapeutic effect for human cervical cancer, brain glioma, bladder cancer, ovarian cancer, melanoma and lung cancer. Also the studies show that wild-type p53 gene can inhibit tumor vascular endothelial growth factor (VEGF) expression so as to reduce tumor vasculogenesis and further promote tumor cell death.

In natural state, polymorphism exists for some amino acid site of wild-type p53 gene. For example, the polymorphism of Porline and Serine occurs at 47 amino acid site of wild-type p53. A study found that wild-type p53 gene with proline (47P) at 47 amino acid site mediated kinase p38 MAPK can make phosphorylation of neighboring serine at 46 amino acid site, and greatly increase its ability to induce cell apoptosis. Reversely, its ability to induce cell apoptosis dropped down 2-5 folds for wild-type p53 gene with serine at 47 amino acid site.

Likewise, existence of common polymorphism of proline (72P)/arginine (72R) at 72 amino acid site of wild-type p53 gene is of great significance for its function of cell apoptosis. Wild-type P53 gene with 72R type has 2 to 15 times in its ability of cell apoptosis higher than 72P type (Murphy M, et al: 2003, 3(3):357-65, Nat. Genetics), wherein the mechanism of wild-type p53 with 72R type is considered at least partly due to the increase of 72R type P53 gene aggregating in the mitochondrion, and to be direct interaction with cell apoptosis protein BAK to destroy mitochondrion and result in cell apoptosis by deficiency of energy.

Recently, a new variety of p53 gene, p53-46F, which is mutated to p53-46F (Phe) from p53-46S (Ser), had been proved more effective than wild-type P53 gene to promote apoptosis in many cancer cells, and its trans-activation level of its downstream genes, including genes Noxa, P53AIP1, and P53RFP, is stronger than dose wild-type P53 (Nakamura Y, et al. Cancer Sci. 2006. July (97(7):633-641).

Beside, researches in recent years also proved that as a downstream gene of P53 gene, P53AIP1 is stronger than wild-type P53 gene in cell apoptosis, wherein the mechanism is considered to be related to its down regulation of mitochondria membrane potential to increase cytochrome C releasing. More importantly, cancer cells expressing wild-type p53 gene has resistance to treatment of exogenous wild-type p53, but P53AIP1 is able to effectively kill the cancer cells expressing wild-type p53 gene. Moreover, to apply both of wild-type P53 and P53AIP1 simultaneously for treatment of cancer cells has synergic effect to induce cell apoptosis (Oda K, et al. Cell 2000. Sep. 15. 102 (6): 849-62; Yoshida K, et al. Cancer Sci. 2004 Jan. 95(1): 91-97; Koschi Matsuda, et al. Cancer Research (2002) 62:2883-2889).

The results of studies described as above has laid a solid foundation for effective gene therapy by application of both of P53 variety and P53AIP1 for treatment of variety of malignant tumors, whatever it is expressing wild-type P53 or expressing mutant P53.

There is no doubt that success or failure of anti-cancer gene therapy depends on the best combination of three major elements for the entire expressing construction. The first element is the adenovirus vector entering into the body cells, which should be safety and high efficient; the second element is the effectiveness of target gene for therapy; the third element is the promoter to drive expression of anti-cancer gene and its tissue/cell specificity.

At present, most of the vectors for delivery of gene into the bodies are replication-deficiency adenovirus vectors. These vectors has following advantages, such as height ability of infection, no matter of target cell being a division or non-division period of target cells, not being integrated into human genome, and producing a large number of high-titer adenovirus and its transient expression, and so on, so that replication-deficiency adenovirus vectors currently become the best choice of the vectors for tumor gene therapy.

There are varieties of origin of promoters to be used for therapy gene of recombinant adenovirus. Currently mCMV promoter is widely used. However, the promoter has almost no specificity of tissue or cell, so it is not so suitable for gene therapy with high toxin.

No matter what, the target-orientated gene therapy is one of important directions for development of gene therapy. Therefore, it is extremely important to identify a tumor-specific promoter.

Recently, a tumor-specific promoter has been identified. Two binding sites have been confirmed in the promoter. One binding site is for an activate protein factor-1 (AP-1), which is a nuclear transcription factor, and another is for polymavirus enhancer activator 3 (PEA-3). The activated protein factor-1 (AP-1) and PEA-3 are over-expressed in most tumor cells. Gene mutation study had proved that to mutate the binding sites or any one binding site as above within the promoter therein will lose its function to drive expression of target gene specifically in the tumor cells, showing that these two binding sites as mentioned above within the promoter is the decisive condition to determine its tumor-specific expression. More importantly, it has been proved that the tumor-specific promoter has a higher activity in tumor cells, and no activity or extremely low activity in the normal cells. Therefore, it was determined as a tumor-specific promoter (Haviv, Y. S., et al: Curr. Gene Ther. Adv, Drug. Delivery Rev. 53, 135-154; Haviv, Y. S., et al: Curr. Gene. Ther. (2003)3,357-365; Su, Z Z. et al. PNAS (2005), 102(4): 1059-1064; Devanand Sarkar, et al. PNAS (2005), 102(39): 14034-14039).

Accordingly, to construct a new generation of recombinant adenoviruse with tumor-specific promoter driving expression of target gene within tumor cells exclusively will provide clinical tumor therapy with an adenovirus of target-oriented tumor gene therapy and therefore is meaningful for the application of tumor gene therapy.

### SUMMARY OF THE PRESENT INVENTION

A main object of the present invention is to provide triple elements, that is a variety of tumor suppressor gene P53 with 72R, p53AIP1 gene (p53-regulated apoptosis inducing protein 1) and a regulation element of tumor-specific promoter, to construct a recombinant adenovirus of target-oriented co-expressing human P53 and P53AIP1, which possess stronger efficiency of anti-cancer, and not damaging the normal cells. So it will become a new generation product for the gene therapy of clinical malignant tumor.

Accordingly, in order to accomplish the above objects, a recombinant adenovirus of target-oriented co-expressing human P53 and P53AIP1 of the present invention is provided, wherein an expression cassette being able to target-oriented co-expressing human P53 and P53AIP1 is inserted into E1 deletion region of the adenovirus, wherein the expression cassette comprises the tumor-specific promoter, the human P53, an internal ribosome entry site IRES, the human P53AIP1, and a SV40 poly-adenylation signal.

Construction of the expression cassette is described as followings:

(a). amino acid of codon 72 of human wild-type p53 suppressor gene is mutated from proline to arginine, wherein the composition and arrangement order of other amino acids are remaining the same;

(b). P53 suppressor gene is located at downstream of tumor specific promoter and upstream of IRES fragment, ligated by Not1 endonuclease site of N-terminal of p53 and EcoR 1 endonuclease site of C-terminal, respectively;

(c). P53AIP1 gene is located at downstream of IRES fragment and upstream of SV40 polyadenylation signal, ligated by Sma1 endonuclease site of N-terminal and Xba1 endonuclease site of p53AIP1, respectively;

(d). The tumor-specific promoter is located at downstream of ES (encapsidation signal of adenoviruse) of pShuttle vector and upstream of P53 gene, ligated by Kpn1 endonuclease site at N-terminal of and Not1 endonuclease site at C-terminal of tumor-specific promoter, respectively.

The expression cassette has the following encoding amino acid sequence and the deoxyribonucleic acid (DNA) sequence having the transcription function:

(a). A tumor-specific promoter: DNA sequence having the transcriptional function; (b). tumor suppressor gene P53: original source from human, and the DNA sequence encoding new variety of human tumor suppressor gene P53, in other words, arginine (R72) replaced proline (72P) of wild-type P53, wherein other encoding amino acid sequences are remaining the same;

(c). P53AIP1 gene: a DNA sequence encoding an apoptosis inducing protein (P53AIP1) regulated by P53 gene from the human; and

(d). IRES (Internal ribosome entry site): a DNA sequence from encephalomyocarditis virus.

Gene driven by the tumor-specific promoter to express and locate at upstream of IRES can be variety of human tumor suppressor gene P53 (72R), or variety of human tumor suppressor gene P53 (46F), which is from a codon 46 serine(S) of wild-type P53 gene being replaced by a phenylalanine (F).

The gene located at downstream of IRES is P53AIP1 gene, or other genes for promoting apoptosis, such as Noxa, P53RFP and P27 (kip 1), or immune regulator such as MDA-7/IL-24, IL-2, IL-6, IFN-γ, or granulocyte/macrophage colony-stimulating factor (GMCSF) and TNF-a.

The tumor-specific promoter is a murine tumor-specific PEG-3 gene promoter, and also can be one of human telomerase promoter, estrogen and hypoxia response promoter, human prostate cancer-specific promoter, or alpha-fetoprotein promoter (AFP).

The recombinant adenovirus is a replication-deficient Ad5 adenovirus, or a conditionally replicating adenovirus. The replication defective Ad5 adenovirus is the product of AdEasy-1 vector of the Stratagene Company, wherein its E1 and E3 region are deleted.

Accordingly, in order to accomplish the above objects, the present invention provides a method of constructing the recombinant adenovirus of target-oriented co-expressing human P53 and P53AIP1, comprising the following steps:

(a). using Endonuclease Pme1 to digest the recombinant plasmid DNA of pShuttle containing expression cassette comprising a tumor specific promoter, a variety of p53, P53AIP1 and SV40 polyadenylation signal, and then electrophoresis and purification for digested plasmid DNA were performed to prepare a linearization recombinant plasmid DNA of pShuttle-P53-P53AIP1;

(b). transforming BJ5183-AD-1-cell, which is bacteria pre-transformed with plasmid pAdEasy-1, with linearized recombinant plasmid DNA of pShuttle-p53-p53AIP1 by electroporation to produce recombinant adenovirus, wherein then these bacteria are screened on LB-kanamycin agar plate, and chosen to find the bacteria strain with resistance-kanamycin, which will be positive bacteria containing recombinant adenovirus vector pAdEasy-1-p53-p53AIP1;

(c). culturing the positive bacteria strains of the smallest size having the recombinant adenovirus pAdEasy-1-p53-p53AIP1 for extraction of the recombinant adenovirus DNA pAdEasy-1-p53-p53AIP1, wherein the DNA is linearized by Pac1 enzyme digestion and purified for later use;

(d). transfecting Embryonal kidney Ad-293 cell with above purified and linearized recombinant adenovirus DNA pAdEasy-1-p53-p53AIP1; and
(e). preparing a primary recombinant adenovirus stock after the transfection of embryonal kidney 293 cell 7-10 days later, and also amplifying the recombinant adenovirus, the Ad-293 cell needs to be transfected with primary recombinant adenoviruse again.

The adenovirus of target-oriented co-expressing p53 and p53AIP1 of the present invention is provided as the gene therapy medicine for the treatment of variant malignant tumor disease, wherein the effective ingredients of the present invention are the variety of human p53 and p53AIP1 protein produced by the adenovirus after transfection of the cells.

The expression construct for expressing a variety of tumor suppressor p53 gene and p53AIP1 gene of the present invention are mainly characterized by 1). 72 codon of wild-type p53 suppressor gene is mutated from proline to arginine, and other amino acid sequence and its arrangement are remaining the same as wild-type p53; 2). expression cassette comprises the tumor-specific promoter, the human P53 (72R), an internal ribosome entry site IRES, the human P53AIP1, and a SV40 polyadenylation signal. Therefore, due to its target-oriented co-expression of double anti-cancerous genes, the adenovirus of the present invention possesses not only stronger anti-cancerous function and wilder anti-cancerous pattern but also has no damage to biological functions of normal cell, and is able to kill malignant tumor cells effectively, so it is available to be applied in tumor gene therapy for kinds of cancers.

These and other objectives, features, and advantages of the present invention will become apparent from the following detailed description, the accompanying drawings, and the appended claims.

### BRIEF DESCRIPTION OF THE DRAWINGS

FIG. 1 is a PCR product of the human wild-type tumor suppressor factor p53 gene:

applying synthetic specific primers and using cDNA of normal human tissue as a template for the PCR reaction, wherein a 1.5% agarose gel electrophoresis is used for the separation of the reactant and for the cloning of the purified reactant. The first and the second lane of the electrophoresis is the production of PCR, and the third lane of the electrophoresis is DNA molecular marker of Φ174/Hae III.

FIG. 2 is a map for endounclease digestion of mutant R72 of the tumor suppressor factor P53:

the R72 mutant type tumor suppressor factor R53 formed a new endonuclease small site at the mutant site, wherein the endonuclease small is used to digest the R72 mutant type and the wild-type tumor suppressor factor P53 respectively, so that the R72 mutant type generates two DNA fragments and the wild-type only generates one DNA fragment.

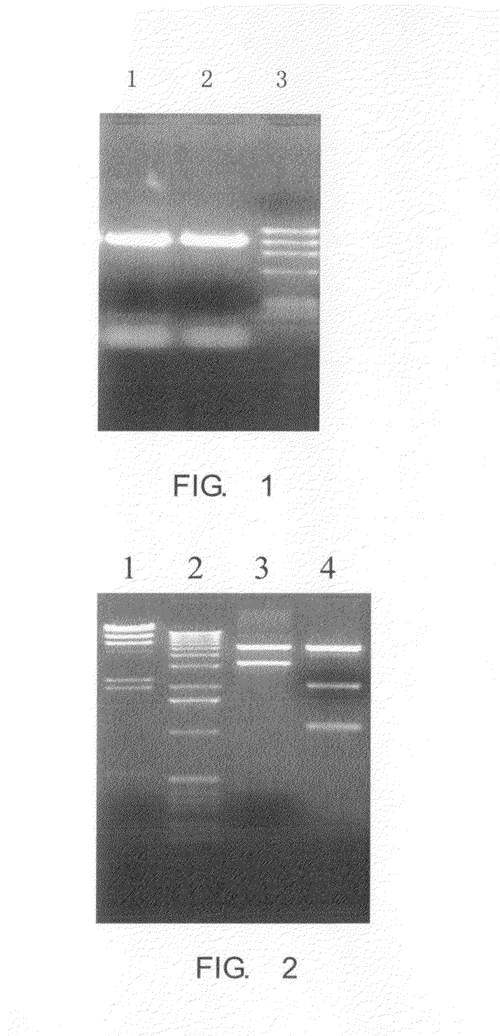

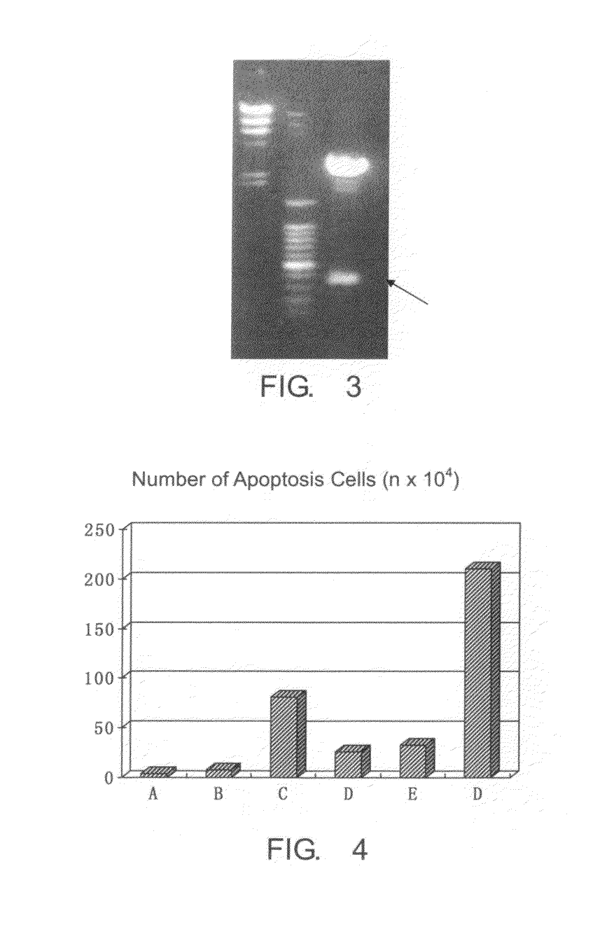

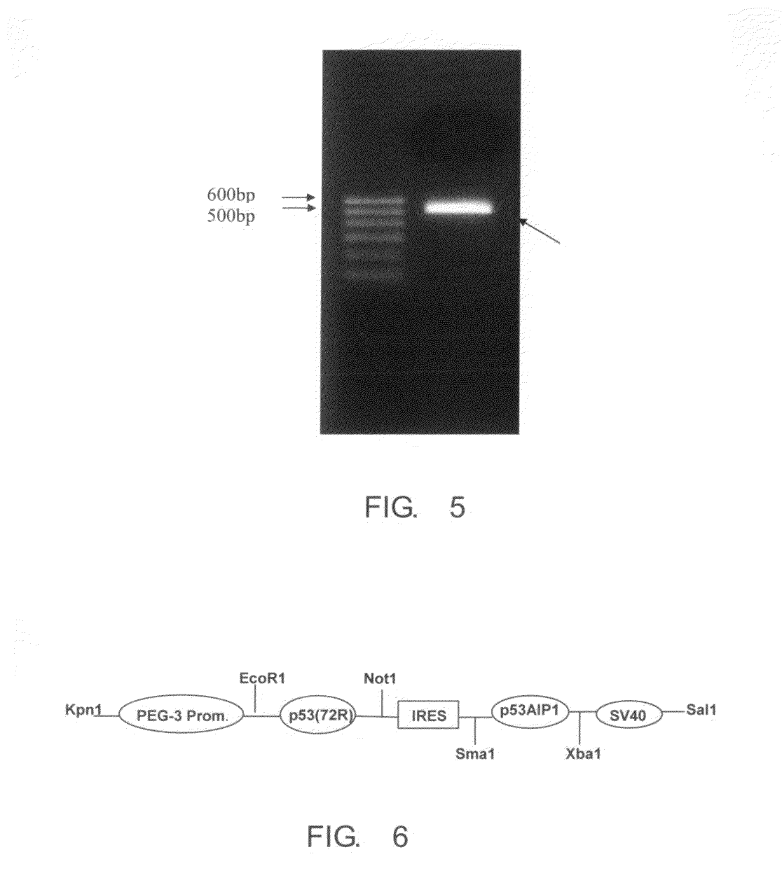

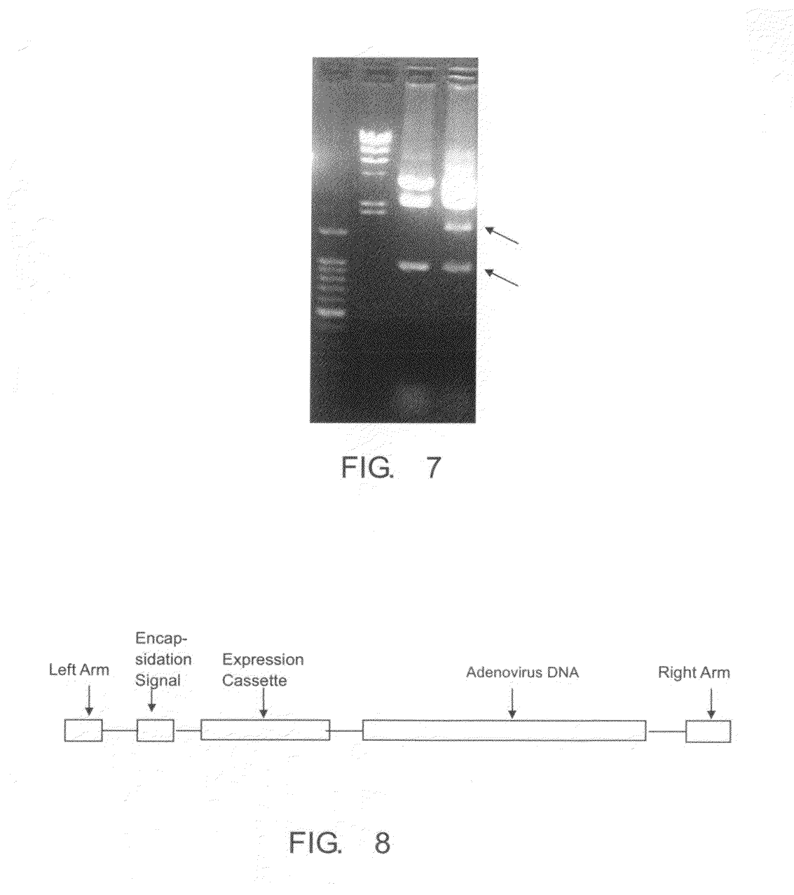

As shown in the FIG. 2 of the drawings, the first lane of the electrophoresis is landa/Hind111 molecular marker, the second land of the electrophoresis is 1 kB DNA molecular market, the third land of the electrophoresis is the wild-type tumor suppressor factor P53, and the fourth land of the electrophoresis is the R72 mutant type tumor suppressor factor P53.

FIG. 3 is the cloning of p53-regulated apoptosis inducing protein 1 gene p53AIP1, and showing a map of the EcoR1 enzyme digestion of P53AIP1.

After the RT-PCR reaction, the p53AIP1 is cloned to the T-Easy vector. The first and second lane of the electrophoresis is the DNA molecular marker, and the third lane of the electrophoresis is the result of EcoR1 digestion of the p53AIP1. The arrow is pointing at the P53AIP1 gene in FIG. 3.

FIG. 4 is the observation of lung cancer cell apoptosis by suppressor gene P53, P53AIP1, and synergetic effect of both P53 and P53AIP1: this is the average result of three transfections of the lung cancer cells by expression plasmid of following genes. A is the control group, B is the wild-type P53, C is the mutant type P53, D is the P53AIP1, E is the wild-type P53+P53AIP1, and F is the mutant type P53+P53AIP1.

FIG. 5 is a cloning of the tumor-specific promoter according to the preferred embodiment of the present invention. This result is from the PCR reactants. The first lane of electrophoresis is the DNA molecular standard, and the second, third, and fourth lanes of the electrophoresis are the reactant from PCR. The arrow on the FIG. 5 is pointing at the reactant from the PCR reaction.

FIG. 6 is a structural map of the expression cassette driven by tumor-specific promoter according to the preferred embodiment of the present invention, illustrating the expression cassette map shows that it's comprised a tumor-specific promoter located at the end of N-terminal, a variety of P53, IRES, P53AIP1, and SV40 polyadenine, and all of these genes and fragments are ligated to each other by the various endonuclease sites.

FIG. 7 is the endonuclease digestion map of expression cassette consisting of a variety of tumor suppressor factor P53, the internal ribosome entry site (IRES), p53AIP1 and SV40 polyadenylation signal.

Recombinant IRES plasmid DNA produced a variety of p53 with full-length (up arrow point to the position), and a DNA fragment comprising IRES of full length and part of p53AIP1 gene (low arrow point to the position) after being digested by BamH1 enzyme.

The first and second lanes are DNA standard marker, and the third lane is just for recombinant IRES plasmid containing p53AIP1 gene and the fourth lane is the IRES plasmid containing both of p53 and p53AIP1.

FIG. 8 is a construction figure of recombinant adenovirus containing the expression cassette driven by the tumor-specific promoter accordingly to the preferred embodiment of the present invention, illustrating the construction figure having two ends, which are a left arm and a right arm of an adenovirus respectively, wherein the expression cassette comprises the tumor-specific promoter, R72 type tumor suppressor factor P53, internal ribosome entry site (IRES), p53AIP1 gene and SV40 polyadenylation signal.

### DETAILED DESCRIPTION OF THE PREFERRED EMBODIMENT

One skilled in the art will understand that the embodiment of the present invention as shown in the drawings and described above is exemplary only and not intended to be limited. An adenovirus vector used in the present invention is the product from Stratagene Company, and not intended to be limited.

Referring to FIGS. 1 and 2, a construction of a R72 type tumor suppressor P53 according to a first preferred embodiment of the present invention is illustrated, wherein the construction adapts a cloning human p53 gene as a template, as shown in FIG. 1, and a gene mutation technology to mutate a proline codon ccc (P72) to a arginine codon cgg (R72) of a codon 72 of a human wild-type p53 gene, so as to form a new nucleotide endonuclease small site in the mutant region. In order to avoid the mutation of the other coding sequence of the wild-type p53 during the mutant procedure, the present invention adapts a sub reaction of PCR, so that a reactant from the PCR sub reaction will be combined to complete the R72 type tumor suppressor p53 construction after determining the sequence of the reactant from the PCR reaction is correct.

A specific operation is as followings:

(1). Amplification of a fragment from Nco1 site of 5′-terminal to codon 72 of human wild-type tumor suppressor p53 gene:

human wild-type tumor suppressor p53 gene is used as the template to amplify the fragment desired by synthetic primers for PCR.

The PCR primer design is described as below:

The First Primer:

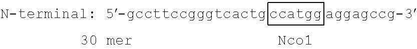

The Second Primer:

Note: ccg is the arginine codon

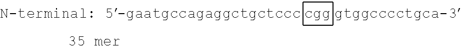

The PCR product is added an adenine by a tailing process, then ligated to plasmid T-easy vector, and then the plasmid is transformed into bacteria for amplification, and the plasmid DNA is extracted from bacteria and purified, and is finally determined by enzyme digest and DNA sequencing.

(2). Amplification of a fragment from the 72 codon to Nco1 site of C-terminal of human wild-type tumor suppressor p53 gene:

human wild-type tumor suppressor p53 gene is used as the template again to amplify the fragment desired by synthetic primers for PCR.

The PCR primer is designed as below:

The third primer: ccg, the proline codon

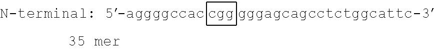

The fourth primer:

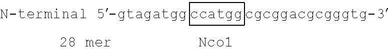

combining the amplified two production of PCR to the T-easy vector as the same method as the step 1), and determining the two products of PCR by the DNA sequencing.

(3). Combining the above two products of PCR of the step (2) to form a Nco1˜Nco1 fragment:

digesting the T-easy vector plasmid DNA of the above DNA fragment of steps (1) and (2), then purifying to combine with the wild-type p53 gene cDNA which is digested by the Nco1 enzyme, and then transforming it into bacteria, extracting the plasmid, and determining the correct recombinant vector by the enzyme digestion to construct the R72 type p53 gene expression plasmid pCMV-neo-p53, as shown in FIG. 2.

Referring to FIG. 3, cloning of p53AIP1 according to a second preferred embodiment of the present invention is illustrated:

1). after 48 hours, collecting the apoptosis cells of Hela tumor cells transfected with expression plasmid of wild-type p53 gene, and then extracting the total nucleotide ribose to synthetic complementary deoxyribonucleic acid (cDNA) through a method of reverse transcriptase.

2). applying the PCR technology to amplify p53AIP1 gene: taking the above synthetic cDNA as a template, and amplifying the PCR by a synthetic primer.

The primer is designed as below:

The first primer: (adding the endonuclease site Sma1 at an end of N-terminal)

5′-ctcccggggatgggatcttcctctgaggcgagcttcaga-3′

The second primer: (adding the endonuclease site Xba1 at an end of C-terminal)

5′-tgagatcttcagttcccagctctgtccaatgctctg-3′

The PCR amplification production is added an adenine by a tailing process, then ligated to a T-easy vector and transformed into bacteria, and then amplified, extracted, purified, and determined the T-easy plasmid having PCR product (p53AIP1) by method of DNA sequencing.

3). ligating the sequencing proved p53AIP1 gene fragment to eukaryotic cell expression plasmid pCMV-neo vector in a correct direction, so as to construct a pCMV-neo-p53AIP1 expression plasmid.

An observation of the tumor suppressor p53, R72 type tumor suppressor p53 and p53AIP1, and a synergy of both of the R72 type tumor suppressor p53 and p53AIP1 to the tumor cell apoptosis according to a third embodiment of the present invention.

The present invention applies the above gene and the gene combination to carry out the research of anti-tumor effects of variable tumor cell lines, including lung cancer H460, cervical cancer Hela, brain glioma, breast cancer, and prostate cancer, in vitro, wherein the research shows that R72 type tumor suppressor p53 and p53AIP1 co-transfection tumor cell has the strongest anti-cancer function. Take the lung cancer H460 for example to describe the third embodiment as below. First, according to the design of this experiment, take a 10% calf serum DMEM medium and under a carbon dioxide incubator with 5% carbon dioxide and 37° C. temperature to culture the lung cancer cells H460 until a Petri dish is 80% covered by the lung cancer cells. Remove the medium and add the new made medium into the Petri dish to culture the cell for another three hours. Add 100 micro-liter, 1.5M salt solution into a sample of plasmid DNA 4 microgram of wild-type p53, R72 type p53, p53AIP1, wild-type p53+p53AIP1, and mutant type p53+p53AIP1 respectively and well mix each of the samples above, according to the description of the jetPEI™ transfection reagent product. At the same time, add 6 micro-liter jetPEI™ transfection reagent into 100 micro-liter 1.5M salt solution and well mix it.

Slowly add the well mixed jetPEI™ transfection reagent into the plasmid DNA salt solution and well mix it. Resting place at the room temperature for 30 minutes, and then drip the transfection reagent containing the plasmid DNA into the cell medium. Take the jetPEI™ transfection reagent as a control group. After culturing for 48 hours, centrifuge to collect the floating lung cancer cells, and count the number of apoptosis cells of the different groups to statistically analyze it. Repeat this experiment for three times to get an average data. The result shows that the ability of the apoptosis of the R72 type p53 is stronger than the wild-type p53, and the R72 type tumor suppressor p53 and p53AIP1 co-transfection of tumor cell have the strongest anti-cancer effect, as shown in FIG. 4.

Referring to FIG. 4, a synthetic and cloning of the tumor-specific promoter DNA fragment according to a fourth embodiment of the present is illustrated.

According to a known gene sequence, the tumor-specific promoter DNA fragment is synthesized, and PCR amplified by the following primers:

The first primer: (adding the endonuclease site Nru1, Kpn1, and Xba1 at the end of N-terminal)

5′-tgtcgcgaggtacctctagaccacggtgacctcacaa-3′

The second primer:

5′-tagatatcacctgggctctcct-3′

The PCR amplification production is added an adenine by a tailing process, then ligated to plasmid T-easy vector and transformed into bacteria, and then amplified, extracted, purified, and determine the PCR product for tumor specific promoter ligated to the plasmid T-easy by DNA sequencing method.

Referring to FIGS. 5 and 6 of the drawings, a construction of an expression cassette comprises the tumor-specific promoter, variety of p53, internal ribosome entry site (IRES), and p53AIP1 according to a fifth embodiment of the present invention is illustrated.

In the fifth embodiment of the present invention, the plasmid pIRES-neo is adapted as a vector for the construction of expression cassette. Through a serious of endonuclease digestion, ligation reaction, transformation of competent bacterial and DNA sequencing, it was confirmed that the construction of the expression cassette was completed. The process includes: 1. take the fragment between Sma1 site of 5′-terminat and Xba1 site of 3′-terminal of the p53AIP1 gene to link to IRES and SV40 polyadenylation signal and located at the downstream of IRES; 2. take the fragment of Not1 site of 5′-terminal and EcoR1 site of 3′-terminal of variety of p53 to insert between tumor-specific promoter and IRES, and locate at the downstream of the promoter; 3. take the fragment between Kpn1 site of 5′-terminal and Not1 site of 3′-terminal of the PEG-3 promoter to insert between the vector with CMV promoter deleted and the variety of p53. Until now the construction of expression cassette of the pIRES plasmid (pPEG-3prom.-p53-IRES-P53AIP1) is completed.

Referring to FIG. 7, a construction of an adenovirus shuttle plasmid containing the expression cassette according to a sixth embodiment of the present invention is illustrated.

1). Digest the pIRES plasmid DNA described as above by enzyme Kpn1 and Sal1 to produce two DNA fragments of Kpn1-Kpn1 and Kpn1-Sal1, which includes the SV40 poly-adenine DNA sequence, and then the digested DNA is separated by 1.2% agarose gel electrophoresis and purified for the later use.

2). Digest adenovirus pShuttle plasmid DNA by enzyme Kpn1 and Sal1, and then digested reactant is separated by 1.2% agarose gel electrophoresis and purified for the later use.

3). Under the effect of ligase enzyme, the above linearization pShuttle plasmid DNA is ligated to Kpn1-Kpn1 and Kpn1-Sal1 fragments produced from digestion of the expression cassette, and the ligated reactant was transformed into competent bacteria.

4). Screening of the bacteria strains, cultivate the transformants, and then extract the recombinant plasmid DNA, and finally to prove it by enzyme digestion and DNA sequencing.

A preparation of a recombinant adenovirus of target-oriented co-expressing variety of p53 and p53AIP1 gene according to a seventh embodiment of the present invention is illustrated:

1). the preparation of linearization recombinant plasmid DNA of pShuttle-p53-p53AIP1 containing expression cassette comprising variety of p53 and p53AIP1 gene: take appropriate amount of the above recombinant shuttle plasmid DNA to be digested by a endonuclease Pme1, separated by the electrophoresis, and purified the linearization recombinant shuttle plasmid DNA for the later use.

2). the above linearization recombinant shuttle plasmid DNA is electroporated into BJ5183-AD-1 bacteria pre-transformed with adenovirus vector pAdEasy-1 for homologous recombination, and the bacteria are screened on the LB-broth containing kanamycin antibiotics, wherein the strains resistance to kanamycin are the bacteria having the recombinant adenovirus pAdEasy-1-p53-p53AIP1.

3). Cultivate the bacteria strain resistance to kanamycin to amplify, and extract recombination adenovirus pAdEasy-1-p53-p53AIP1 DNA from the bacteria, and then linearize the recombination adenovirus pAdEasy-1-p53-p53AIP1 plasmid DNA by digestion with enzyme Pac1, and purify it for later use.

4). The linearized recombination adenovirus pAdEasy-1-p53-p53AIP1 plasmid DNA is used to transfect embryonic kidney AD-293 cell to allow the recombination adenovirus pAdEasy-1-p53-p53AIP1 to be packaged to make it become infectious viral particles.

5). Preparation of primary recombinant adenovirus stock with recombinant adenovirus pAdEasy-1-p53-p53AIP1 by removing growth medium from adenovirus-producing AD-293 cell plate after the transfection of embryonic kidney AD-293 cell for 7-10 days, and obtaining adequate amount of the high-titration recombinant adenovirus by infection of the embryonic kidney AD-293 cells with a low passage viral stock.

6). Increase adequate among of the high-titration recombinant adenovirus.

It will thus be seen that the objects of the present invention have been fully and effectively accomplished. The embodiments have been shown and described for the purposes of illustrating the functional and structural principles of the present invention and is subject to change without departure from such principles. Therefore, this invention includes all modifications encompassed within the spirit and scope of the following claims.

1. A recombinant adenovirus of target-oriented co-expressing a variety of human p53 and p53AIP1 gene, wherein an expression cassette construction of target-oriented co-expressing human p53 and p53AIP1 is inserted in the E1 deletion region of said recombinant adenovirus, wherein said expression cassette comprises a tumor-specific promoter, a variety of human p53, a internal ribosome entry site IRES, a human p53AIP1, and a SV40 polyadenylation signaline.

2. The recombinant adenovirus of target-oriented co-expressing a variety of human p53 and p53AIP1 gene, as recited in claim 1, wherein a construction of said expression cassette comprises:
a) amino acid of codon 72 of human wild-type p53 suppressor gene being mutated from proline to arginine, wherein the composition and arrangement order of the other amino acids of said human wild-type p53 are remaining the same;
b) N-terminal and C-terminal end of said variety of human wild-type p53 suppressor gene are inserted into upstream of said internal ribosome entry site and downstream of said tumor specific promoter by enzyme Not1 site and enzyme EcoR1 site fragment;
c) N-terminal end and C-terminal end of said p53AIP1 gene are inserted into downstream of said internal ribosome entry site and SV40 polyadenylation signal by Sma1 site and Xbal1 site fragment; and
d) N-terminal end and C-terminal end of said tumor-specific promoter are inserted into upstream of said p53 gene and downstream of adenovirus ES (encapsidation signal) by Kpn1 site and Not1 site fragment.

3. The recombinant adenovirus of target-oriented coexpressing human p53 and p53AIP1, as recited in claim 1, wherein the said expression cassette comprises coding amino acid sequence and sequence of deoxyribonucleic acid (DNA) having transcription function which comprises:
a) said tumor-specific promoter, wherein said tumor-specific is a DNA sequence having said transcription function;
b) source of said p53 gene being from human and said deoxyribonucleic acid sequence coding amino acid of the variety of tumor suppressor p53 gene (R72) which is 72 codon proline of human wild-type p53 being replaced by codon arginine, wherein other coding sequences of variety of human tumor suppressor p53 has the same coding sequence as wild-type p53 gene;
c) coding a deoxyribonucleic acid sequence of p53 regulated apoptosis inducing protein 1 (p53AIP1) gene from human being; and
d) said internal ribosome entry site (IRES) deoxyribonucleic acid sequence, which is from encephalomyocarditis virus.

4. The recombinant adenovirus of target-oriented co-expressing human p53 and p53AIP1, as in claim 1, wherein said gene driven by the tumor-specific promoter to co-expressing and located at upstream of said IRES is said variety of human suppressor p53 gene (72R), or a variety of human suppressor p53 (46F), wherein said human suppressor p53 (46F) has a 46 codon phenylalanine which replaced the 46 codon serine of said wild-type p53.

5. The recombinant adenovirus of target-oriented co-expressing human p53 and p53AIP1, as in claim 1, wherein said gene located at downstream of said IRES is one of said p53AIP1 gene, other apoptosis gene, an immune regulator, and granular/macrophages colony-stimulating factor (GMCSF) and TNF-a.

6. The recombinant adenovirus of target-oriented coexpressing human p53 and p53AIP1, as in claim 1, wherein said tumor-specific promoter is one of a murine tumor-specific PEG-3 gene promoter, a human telomerase promoter, a estrogenic hormone and hypoxia response promoter, human prostate cancer-specific promoter, and alpha-fetoprotein promoter (AFP).

7. The recombinant adenovirus of target-oriented co-expressing human p53 and p53AIP1, as recited in claim 1, wherein an adenovirus vector is one of a replication deficiency Ad5 adenovirus and a conditionally replication adenovirus vector.

8. The recombinant adenovirus of target-oriented co-expressing human p53 and p53AIP1, as recited in claim 1, wherein said replication deficiency adenovirus is that E1 and E3 region of adenovirus vector are deleted.

9. A method for preparing a recombinant adenovirus of target-oriented co-expressing human p53 and p53AIP1, comprising the steps of:
(a) preparing a linearized recombinant shuttle plasmid DNA pShuttle-p53-p53AIP1, wherein a recombinant shuttle plasmid pShuttle-p53-p53AIP1 containing an expression cassette co-expressing a variety of human p53 and p53API1 gene by tumor specific promoter is digested by endonuclease pMe1, and separated by electrophoresis, and purified for preparation of said linearized recombinant shuttle plasmid pShuttle-p53-p53AIP1;
(b) electroporating the linearized recombinant shuttle plasmid DNA into a competent bacteria for homologous recombination with adenovirus vector, wherein the bacteria is pre-transformed with adenovirus vector pAdEasy-1, and the transformed bacteria is screened on the LB-broth plates containing kanamycin antibiotics, wherein the bacteria strains resistance to kanamycin are the bacteria having the adenovirus pAdEasy-1 recombinant;
(c) picking up the bacteria strains resistance to kanamycin to cultivate, wherein the adenovirus pAdEasy-1 recombinant DNA is extracted from the bacteria, digested by Pac1 enzyme to be a linear recombinant adenovirus pAdEasy-1 DNA, and then separated on argrose gel by electrophoresis and purified for later use;
(d) transfecting embryonic kidney AD-293 cell with linearized recombinant adenovirus pAdEasy-1 DNA for being packaged to become infectious viral particles, wherein the embryonic kidney AD-293 cell transformed by sheared adenovirus type 5 DNA can produce the adenovirus E1 gene in trans for packaging linearized recombinant adenovirus pAdEasy-1 and making it infectious viral particles; and
(e) preparing a primary recombinant adenovirus stock after the transfection of AD-embryonic kidney 293 cell 7-10, and optimizing of the conditions for primary recombinant adenovirus stock to transfect the embryonic kidney AD-293 cell for amplification of the recombinant adenovirus.

10. The recombinant adenovirus of target-oriented co-expressing human p53 and p53AIP1, as recited in claim 1, wherein said recombinant adenovirus of target-oriented co-expressing human p53 and p53AIP1 is provided as an application of gene therapy for treatment of a plurality of malignant cancer disease, wherein an effective ingredient of said recombinant adenovirus is co-expressing proteins for variety of human p53 and p53AIP1 gene of said recombinant adenovirus after infectious of cells.

11. The recombinant adenovirus of target-oriented co-expressing human p53 and p53AIP1, as in claim 2, wherein said gene driven by the tumor-specific promoter to co-expressing and located at upstream of said IRES is said variety of human suppressor p53 gene (72R), or a variety of human suppressor p53 (46F), wherein said human suppressor p53 (46F) has a 46 codon phenylalanine which replaced the 46 codon serine of said wild-type p53.

12. The recombinant adenovirus of target-oriented co-expressing human p53 and p53AIP1, as in claim 2, wherein said gene located at downstream of said IRES is one of said p53AIP1 gene, other apoptosis gene, an immune regulator, and granular/macrophages colony-stimulating factor (GMCSF) and TNF-a.

13. The recombinant adenovirus of target-oriented coexpressing human p53 and p53AIP1, as in claim 2, wherein said tumor-specific promoter is one of a murine tumor-specific PEG-3 gene promoter, a human telomerase promoter, a estrogenic hormone and hypoxia response promoter, human prostate cancer-specific promoter, and alpha-fetoprotein promoter (AFP).

14. The recombinant adenovirus of target-oriented co-expressing human p53 and p53AIP1, as in claim 3, wherein said gene driven by the tumor-specific promoter to co-expressing and located at upstream of said IRES is said variety of human suppressor p53 gene (72R), or a variety of human suppressor p53 (46F), wherein said human suppressor p53 (46F) has a 46 codon phenylalanine which replaced the 46 codon serine of said wild-type p53.

15. The recombinant adenovirus of target-oriented co-expressing human p53 and p53AIP1, as in claim 3, wherein said gene located at downstream of said IRES is one of said p53AIP1 gene, other apoptosis gene, an immune regulator, and granular/macrophages colony-stimulating factor (GMCSF) and TNF-a.

16. The recombinant adenovirus of target-oriented coexpressing human p53 and p53AIP1, as in claim 3, wherein said tumor-specific promoter is one of a murine tumor-specific PEG-3 gene promoter, a human telomerase promoter, a estrogenic hormone and hypoxia response promoter, human prostate cancer-specific promoter, and alpha-fetoprotein promoter (AFP).

17. The recombinant adenovirus of target-oriented co-expressing human p53 and p53AIP1, as recited in claim 1, wherein said recombinant adenovirus of target-oriented co-expressing human p53 and p53AIP1 is provided as an application of gene therapy for treatment of a plurality of malignant cancer disease, wherein an effective ingredient of said recombinant adenovirus is co-expressing proteins for variety of human p53 and p53AIP1 gene of said recombinant adenovirus after infectious of cells.


SEQUENCE LISTING (6 sequences)

SEQ ID NO: 1 - DNA, 1701 bp, Artificial
  Nucleotide sequences of p53 mutated from proline to arginine at 72 amino acid of N-terminal
atcgatatcgccaccgtccagggagcaggtagctgctgggctccggggacactttgcgttcgggctgggagcgtgctttccacgacggtgacacgcttccctggattggcagccagactgccttccgggtcactgccatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccgggtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcatgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtagggacccacttcaccgtactaaccagggaagctgtccctcactgttgactagtaattcgcggccgcgtcg

SEQ ID NO: 2 - protein, 393 aa, Artificial [sequence omitted]

SEQ ID NO: 3 - DNA, 591 bp, Artificial
  Nucleotide sequences for promotor of PEG-3 gene derived from Rat
tgtcgcgaggtacctctagaccacggtgacctcacaaggcccggaatctccgcgagaatttcagtgttgttttcctctctccacctttctcagggacttccgaaactccgcctctccggtgacgtcagcatagcgctcgtcagactataaactcccgggtgatcgtgttggcgcagattgactcagttcgcagcttgtggaagattacatgcgagaccccgcgcgactccgcatccctttgccgggacagcctttgcgacagcccgtgagacatcacgtccccgagccccagcctgagggcgacatgaacgcgctggccttgagagcaatccggacccacgatcgcttttggcaaaccgaaccggaccgaaccggacctccagcccccggggtgacgcgcagtcgccggtgagtgggggatggggcggcctttgggggagtgctggggaggactttctttggcgatggaggctaggagagtgttgtgggatctaggggagactggggaggaacccagatttgaggaaacggcactgaaagccggatgctttatttggtccgagagaggagagcccaggtgatagatatcta

SEQ ID NO: 4 - DNA, 370 bp, Artificial
  nucleotide sequences for human p53AIP1 gene
tggacccccgggctggatcctgatgggatcttcctctgaggcgagcttcagatctgctcaagcttcctgcagtggggccaggaggcagggcctgggcaggggagaccagaacctctcggtgatgcctccgaatggcagggctcagacacacacacctggctgggtaagtccctgcagtgaaaaccgagacggtcttttgcctgccacagccccgggcagactctgctctcaccgtggtgccgacatcccaagttttcagactcaccaggacccagtgacagcatctgggtcctcagagctgcatgcggactgtccccagttcagagcattggacagagctgggaactgacagaattccatctagacggta

SEQ ID NO: 5 - protein, 108 aa, Artificial
  Amino acid sequences for protein of human p53AIP1 gene
MGSSSEASFRSAQASCSGARRQGLGRGDQNLSVMPPNGRAQTHTPGWVSPCSENRDGLLPATAPGRLCSHRGADIPSFQTHQDPVTASGSSELHADCPQFRALDRAGN

SEQ ID NO: 6 - DNA, 3573 bp, artificial
  Epression cassette consisting of  sequences of PEG-3 promotor, p53(72R) gene , IRES fragment, and p53AIP1gene
tgtcgcgaggtacctctagaccacggtgacctcacaaggcccggaatctccgcgagaatttcagtgttgttttcctctctccacctttctcagggacttccgaaactccgcctctccggtgacgtcagcatagcgctcgtcagactataaactcccgggtgatcgtgttggcgcagattgactcagttcgcagcttgtggaagattacatgcgagaccccgcgcgactccgcatccctttgccgggacagcctttgcgacagcccgtgagacatcacgtccccgagccccagcctgagggcgacatgaacgcgctggccttgagagcaatccggacccacgatcgcttttggcaaaccgaaccggaccgaaccggacctccagcccccggggtgacgcgcagtcgccggtgagtgggggatggggcggcctttgggggagtgctggggaggactttctttggcgatggaggctaggagagtgttgtgggatctaggggagactggggaggaacccagatttgaggaaacggcactgaaagccggatgctttatttggtccgagagaggagagcccaggtgatagatatcgccaccgtccagggagcaggtagctgctgggctccggggacactttgcgttcgggctgggagcgtgctttccacgacggtgacacgcttccctggattggcagccagactgccttccgggtcactgccatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccgggtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcatgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtagggacccacttcaccgtactaaccagggaagctgtccctcactgttgactagtaattcgcggccgcatagataactgatccagtgtgctggaattaattcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactctttttgttgtcaagcttgaggtgtggcaggcttgagatctggccatacacttgagtgacaatgacatccactttgcctttctctccacaggtgtccactctttttgttgtcaagcttgaggtgtggcaggcttgagatctggccatacacttgagtgacaatgacatccactttgcctttctctccacaggtgtccaccctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggctggatcctgatgggatcttcctctgaggcgagcttcagatctgctcaagcttcctgcagtggggccaggaggcagggcctgggcaggggagaccagaacctctcggtgatgcctccgaatggcagggctcagacacacacacctggctgggtaagtccctgcagtgaaaaccgagacggtcttttgcctgccacagccccgggcagactctgctctcaccgtggtgccgacatcccaagttttcagactcaccaggacccagtgacagcatctgggtcctcagagctgcatgcggactgtccccagttcagagcattggacagagctgggaactgacagaattccatctagacggta